(19) United States
(12) Patent Application Publication
(10) Pub. No.: US 2013/0090685 A1
(43) Pub. Date: Apr. 11, 2013

(54) METHODS AND SYSTEMS FOR TISSUE FASTENING

(71) Applicants: Donald A. Gonzales, San Antonio, TX (US); Gabriele G. Niederauer, San Antonio, TX (US); Kevin L. Ohashi, Jamaica Plain, MA (US); Anthony Natale, New York, NY (US); Fred B. Dinger, San Antonio, TX (US)

(72) Inventors: Donald A. Gonzales, San Antonio, TX (US); Gabriele G. Niederauer, San Antonio, TX (US); Kevin L. Ohashi, Jamaica Plain, MA (US); Anthony Natale, New York, NY (US); Fred B. Dinger, San Antonio, TX (US)

(21) Appl. No.: 13/395,294

(22) PCT Filed: Aug. 25, 2010
(86) PCT No.: PCT/US10/46592

§ 371 Date: Dec. 18, 2012

Related U.S. Application Data
(60) Provisional application No. 61/239,677, filed on Sep. 3, 2009.

(51) Int. Cl. A61B 17/08; A61B 17/04

(52) U.S. Cl. CPC A61B 17/083; A61B 17/0401; A61B 17/08
USPC 606/221; 606/232; 606/213

(57) ABSTRACT

Fastening devices are provided that are designed to effectively close tissue as well as instruments for applying the inventive fastening devices. The devices are useful for closing tissue such as mucosa in the oral cavity, oropharynx, hypopharynx, laryngeal surfaces, oronasopharynx, or other mucosal tissues. In particular, uvulopalatopharyngoplasty (UPPP), uvulopalatal flap (UPF) technique, and tonsillectomy can be assisted using the inventive system. Embodiments also provide methods of using the fastening devices and/or instruments, and kits including the fastening devices.

### CROSS-REFERENCE(S) TO RELATED APPLICATION(S)

This application claims priority to U.S. Provisional Patent Application Ser. No. 61/239,677, filed Sep. 3, 2009, the entire contents of which are expressly incorporated by reference

### BACKGROUND INFORMATION

There are over 750,000 tonsillectomies performed each year in the U.S., and 1.5 million performed worldwide. The most common complications of tonsillectomy are pain and bleeding. Post-operative pain frequently leads to hospitalization or at least a visit to a physician due to the pain per se and/or dehydration caused by decreased oral intake due to the pain. Post-operative bleeding also occurs in approximately 5% of all cases. The occurrence of bleeding is bimodal, and bleeding is frequently observed at post-operative day 1 and post-operative days 5-9. Although, historically, tonsillectomy was primarily performed to treat recurrent infection (tonsillitis), 77% of tonsillectomies are now performed to treat sleep-disordered breathing (SDB) Friedman et al., Otolaryngol. Head and Neck Surg. 140(6):800-8. Sleep disordered breathing is a form of obstructive sleep apnea (OSA) in the pediatric population. Although tonsillectomy alone has shown some success in treating SDB, studies have found additional measures need to be performed to treat a growing number of patients, specifically closure of the tonsillar pillars after the removal of the tonsils. There is evidence that closing the tonsillar pillars (e.g., via suturing) improves sleep apnea scores after tonsillectomy, leads to better healing, and reduces post-operative pain. Nandapalan et al., Clin. Otolaryngol. Allied Sci. 20(2):127-29, April 1995; Genc et al., Int. J. Pediatr. Otorhinolaryngol. 70(4):725-30, April 2006; Guilleminault et al. Laryngoscope. 114 (1): 132-7. each of which is incorporated herein by reference. Furthermore, closing of the tonsillar pillars is currently performed in conjunction with uvulopalatopharyngoplasty (UPPP) to treat adults with sleep apnea. Although closing of the tonsillar pillars has been shown to be advantageous, tonsillar pillar dehiscence is a problem after UPPP with tonsillectomy. The overall incidence of dehiscence is approximately 40% and has been found to be independent of the dissection method (i.e., cold scalpel versus electocautery). Altman et al., Laryngoscope 114(2):294-6, February 2004; incorporated herein by reference. Because of this high rate of dehiscence and the amount of surgical time needed to close the tonsillar pillars via suturing, it is not recommended in all tonsillectomies.

Furthermore, mucosal suturing dehiscence is not isolated to the tonsillar pillars, but is seen throughout the digestive tract, specifically closures under tension. High dehiscence rates are seen in closure of oral, pharyngeal, laryngeal, esophageal, and intestinal tissue.

Surgical sealants such as fibrin-based glues have also been used in tonsillectomy as an effective substitute for electrocautery. Sealant use has been shown to provide effective hemostasis and sealing. Vaiman et al., Ann. Otol. Rhinol Laryngol. 112(5):410-14, May 2003; incorporated herein by reference. However, sealant does not remain adherent for the amount of time necessary for the tissue to heal.

### SUMMARY

Embodiments of the present invention may be used to address a need in the art for an effective system for closing mucosa, particularly mucosa of the oral cavity, oronasopharynx, hypopharynx, laryngeal, esophageal and intestinal surfaces. Such a system preferably provides for the effective closure of mucosal tissues without dehiscence. The system may be used in surgical procedures of the oronasopharynx (e.g., UPPP, tonsillectomy, uvulopalatal flap (UPF) technique, dental procedures, laryngectomy, pharyngectomy, esophagectomy, tumor removal, etc.). Embodiments of the present invention provide such devices for the effective closure of mucosa as well as instruments for the delivery of these novel fastening devices and methods of using such devices and/or instruments. Embodiments of the present invention save time and can lead to better outcomes than current suturing techniques. For example, embodiments of the invention may reduce dehiscence rates, reduce bleeding, reduce pain, speed healing, reduce surgical time, and/or improve sleep apnea score results.

In one aspect, the invention provides fastening devices for closing tissues, particular mucosal tissues. Mucosa is typically difficult to suture given its propensity to tear. Any tension may lead to tearing of the suture material through the mucosa secondary to the cross-sectional round geometry of the suture. Because of the round shape, the tension on the suture is concentrated in a minimal area. The inventive fastening device is designed to prevent the tearing of mucosa by the fastening device. In certain embodiments, the device may include flat, elongated, or rectangular cross-sectional shapes of the fastening device to avoid or minimize tearing that is frequently seen with round suture material. The thickness of the material used to construct the closing device may range from approximately 0.5-2 mm. The fastening device may be an angled staple, a curved (e.g., V-shaped, L-shaped, C-shaped, U-shaped, D-shaped) staple or clamp, or a rivet. In certain embodiments, the fastening devices include a mechanism for fastening the device in a closed position. For example, the fastening device may have a male and female part for fastening the device. The male and female parts may be connected by a curved member or two or more members connected at angle(s). When the male and female parts are brought together, the fastening device forms a closed ring-like structure for approximating two or more tissues. The male end may be sharp or pointed in order to penetrate mucosa or other tissues. The male end may also include a flange, swelling, or other feature to lock into the female end of the device. This locking mechanism prevents the devices from easily coming apart after placement. The female end may include a hole, aperture, or cavity for receiving the male end. The fastening of the fastening device may be irreversible or reversible. The device may be typically approximately 0.25-0.75 cm long by 0.25-0.75 wide. In certain embodiments, the device is approximately 0.5 cm by approximately 0.5 cm. As will be appreciated by one of skill in the art, the fastening devices may come in a variety of sizes for use in different applications or different subjects. For example, smaller sized fastening devices are preferable for use in the pediatric population.

In other embodiments, the fastener may be a partial ring that may be ratcheted closed upon placement through the tissue. Other embodiments include a fastener with a solid, molded piece that is secured to the mucosal tissue that is attached to suture.

The device may be either a one piece or a two-piece system. In certain embodiments, the device is a molded, one piece fastening device. The device may be made of a resorbable or non-resorbable material (e.g., a polymeric material). In certain embodiments, the device is made of a resorbable polymeric material such as a polyester. In certain embodiments, the device is made of a resorbable material such as poly(lactic-co-glycolic acid).

The device may be used anywhere in the body of a subject; however, it is particularly useful in the oral cavity, oronasopharynx, and hypopharynx of the head and neck. In certain embodiments, the device is particularly suited for closing the tonsillar pillars in a uvulopalatalpharyngoplasty (UPPP) and/or tonsillectomy. In certain embodiments, the device is particularly suited for use in a uvulopalatal flap (UPF) procedure. In certain embodiments, the device is suited for use in a dental procedure such as tooth extraction. The device may also be used in surgeries involving the gastrointestinal and genitourinary systems.

The inventive fastening devices may be optionally coated. The coating may include a timed release formulation of a pharmaceutical agent such as an anti-inflammatory agent, a steroid, antibiotic, anesthetics, pain reliever, hemostatic agent, etc. The device may also be coated to make the device more biocompatible. Many coatings for medical devices are known in the art and may be applied to the inventive fastening devices.

Embodiments of the invention not only provide fastening devices but also provide instruments for holding and applying the inventive fastening devices to close or approximate two or more tissues. The instrument may include a space for holding a plurality of fastening devices that will be needed in a given surgery or procedure. The instrument may include a handle for comfortable gripping of the instrument. The apparatus works by applying the fastening device to the mucosa or other tissues to be joined and fastening the device. In certain embodiments, the instrument is designed for use in surgical procedures of the head and neck such as the oral cavity, throat, hypopharynx, or oronasopharynx. The instrument is preferably disposable or suitable for sterilization and re-use.

In another aspect the invention provides methods of using the fastening devices and/or the instruments. The devices and instruments may be used in a variety of surgeries or procedures. In certain embodiments, the surgeries or procedures involve the approximation of mucosal surfaces of the head and neck. In certain embodiments, the surgeries or procedures involve the oral cavity, oropharynx, hypopharynx, throat, or laryngeal surfaces. In certain embodiments, the surgery or procedure is one typically performed by a certified physician or other health care professional. In certain embodiments, the surgery is a UPPP. In other embodiments, the surgery is a tonsillectomy. For example, embodiments of the invention provide a method of closing the tonsillar pillars using the inventive fastening devices and apparatuses. In certain embodiments, a sealant, such as a fibrin based product, chitosan based product, thrombin based products, alpha-cellulose based products, collagen based products, albumin based products can be used in conjunction with the fastening devices in order to reduce pain, reduce bleeding, and/or otherwise improve the outcome. In certain embodiments, a coating, such as protein or growth factor based products, can be used in conjunction with the fastening devices in order to enhance healing and/or otherwise improve the outcome. In certain embodiments the surgery is a tonsillectomy and the sealant is placed on the tonsillar bed. In certain embodiments, the surgery is a UPF procedure. In certain embodiments, the surgery is a removal of a tumor of the head or neck. In certain embodiments, the surgery is a dental or oral surgery. In certain embodiments, the surgery involves the closing of laryngeal or pharyngeal defect. The devices and instruments may also be used in other areas of the body including the gastrointestinal tract and genitourinary system. The devices and instruments may also be used in neurosurgery such as dural closure.

In another aspect, the invention provides a kit including the inventive fastening devices. The kit may include multiple sizes of the fastening devices, pharmaceutical agents (e.g., anesthetics, antibiotics), an instrument for applying/fastening the fastening devices, an instrument for removing the devices, instructions for using the fastening devices, etc. The items in the kit may be conveniently packaged for the use by a treating physician. In certain embodiments, the items are sterilely packaged.

One of the many advantages of the inventive system is that it offers physicians an alternative to suturing, which has the problem of a high rate of dehiscence, bleeding, pain, and other complications. The inventive system allows for the efficient closure of mucosa, particularly in the oronasopharynx, the oral cavity, oropharynx, hypopharynx, and laryngeal surfaces. Overall, the inventive system can reduce surgical time, thereby reducing the time a subject is under anesthesia, and improves surgical outcomes.

Exemplary embodiments may comprise a one piece, molded fastening device for use in closing tissue comprising a male fastening feature attached through a member to a female fastening feature, whereby when the male and female fastening features are brought together, the device is able to approximate two tissues by forming a closed ring-like structure. In certain embodiments, the device is suitable for approximating mucosal tissue. The device may be D-shaped, U-shaped, V-shaped, C-shaped, D-shaped, oval, circular, or semi-circular in certain embodiments. In certain embodiments, the device comprises at least two members connected together at an angle.

Exemplary embodiments of the device comprise: a first member with a male fastening feature; a second member with a female fastening feature; and a third member wherein the third member joins the first and second members at the opposite ends of the fastening features; and wherein the first and second member are connected at an angle, and the second and third member are connect at an angle. The three members may form a C-shape or a U-shape. The device may comprise a first member with a male fastening feature and a second member with a female fastening feature, where the first and second member are connected at an acute angle. In specific embodiments, the two members form a V-shape. In certain embodiments, the device is C-shaped with a male fastening feature on one end and a female fastening feature on the opposite end.

In specific embodiments, the device may be approximately 0.2 cm to approximately 1 cm wide, more specifically approximately 0.25 cm to approximately 0.75 cm wide, or more specifically approximately 0.5 cm wide. In specific embodiments, the device may be approximately 0.2 cm to approximately 1 cm long, more specifically approximately 0.25 cm to approximately 0.75 cm long, more specifically approximately 0.5 cm long. In specific embodiments, the device may be approximately 0.2 cm to approximately 1 cm long, and approximately 0.2 cm to approximately 1 cm wide. In specific embodiments, the device may be approximately 0.5 cm long, and approximately 0.5 cm wide.

In specific embodiments, the cross-sectional shape of at least one member is not round. In certain embodiments the cross-sectional shape of at least one member may be flattened, elongated, rectangular, or square. In specific embodiments, the male fastening feature is pointed or sharp. In certain embodiments, the male fastening feature comprises a locking mechanism for fastening the male and female fastening features. In certain embodiments, the female fastening feature is capable of accepting the male fastening feature.

In certain embodiments, the device is made of a biocompatible material. In specific embodiments, the device is made of a biocompatible polymer. In specific embodiments, the device is made of a biocompatible, bioresorbable material. In certain embodiments, the biocompatible, bioresorbable material is a polyester, a polyanhydride, a polyphosphazene, a polyacrylate, or a polymethacrylate. In certain embodiments, the device is made of poly(lactic-co-glycolic acid) (PLGA). In specific embodiments, the device is made of poly(L-lactic-co-glycolic acid) (90% glycolide:10% L-lactide). In specific embodiments, the biocompatible, bioresorbable material is absorbed in approximately 1-4 weeks during use. In specific embodiments, the biocompatible, bioresorbable material is absorbed in approximately 4-6 weeks during use. In other embodiments, the biocompatible, bioresorbable material is absorbed in approximately 6-8 weeks during use. In other embodiments, the biocompatible, bioresorbable material is absorbed in approximately 2-3 months during use.

In certain embodiments, the device is made of a non-biodegradable material. In certain embodiments, the device is coated. In specific embodiments, the device is coated with Teflon (PTFE). In other embodiments, the device is coated with hyaluronidate. In certain embodiments, the device is coated with a polymer, and in specific embodiments the coating comprises a pharmaceutical agent. In specific embodiments, the pharmaceutical agent is selected from the group consisting of hemostatic agents, anesthetics, pain relievers, anti-inflammatory agents, and antibiotics.

Certain embodiments comprise a two piece, rivet-like fastening device for use in closing tissue comprising: a first part with a pointed, male fastening feature substantially perpendicular to a flat surface; and a second part with a female fastening feature substantially perpendicular to a flat surface; whereby when the two parts with their male and female fastening features are brought together, the device is able to approximate two tissues. In specific embodiments, the flat surface is circular or polygonal and approximately 0.2 cm to approximately 1 cm in diameter. Certain embodiments may also comprise a cassette comprising a plurality of fastening devices of any one of claim 1-40 and a package for holding the devices.

Exemplary embodiments may also comprise an instrument for applying a fastening device, where the instrument comprises: a handle and a mechanism for holding and fastening the fastening device. In certain embodiments, the instrument may further comprise a plurality of fastening devices. The instrument may further comprising a cassette comprising a plurality of fastening devices, in certain embodiments. In certain embodiments, the instrument is disposable, while in other embodiments, the instrument may be re-useable. In exemplary embodiments, the instrument can be sterilized after each use.

Exemplary embodiments may also comprise a method of applying a fastening device, the method comprising steps of: approximating two tissues; piercing the first tissue with fastening device; piercing the second tissue with the fastening device; and fastening closed the fastening device. In certain embodiments, any of the steps is aided by the previously-described instrument. In certain embodiments, at least one of the tissues is a mucosal tissue, and in specific embodiments, both the first and second tissue are mucosal tissues. In certain embodiment, the steps are repeated at least 3 times, 5 times, or 10 times. In certain embodiments, the steps are performed in the context of a uvulopalatopharyngoplasty (UPPP) with or without tonsillectomy procedure. In other embodiments, the steps are performed in the context of a uvulopalatal flap (UPF) procedure. In other embodiments, the steps are performed in the context of removal of a tumor. In still other embodiments, the steps are performed in the context of closing a laryngeal or pharyngeal defect. In still other embodiments, the steps are performed in the context of closing an Eustachian tube orifice. In certain embodiments, the steps are performed in the context of a dental procedure. In specific embodiments, the method further comprises applying a tissue sealant to the approximated tissues. In certain embodiments, the tissue sealant is a fibrin-based sealant, while in other embodiments, the tissue sealant is a chitosan based product, thrombin based product, alpha-cellulose based product, collagen based product, or albumin based product.

The inventive fastening devices for use in approximating mucosa or other tissues are designed to reduce or prevent tearing of the tissue or other damage to the tissue. Although suited for use in closing or approximating mucosal tissues, the fastening devices may be used in other non-mucosal tissues. Embodiments of the device may also be used to approximate a mucosal surface to a non-mucosal surface; to approximate a mucosal surface to non-mucosal surface; or to approximate a non-mucosal surface to a non-mucosal surface. Embodiments of the fastening device may be of a variety of shapes and sizes. In certain embodiments, the device is angled, curved, semi-circular, oval, C-shaped, V-shaped, U-shaped, L-shaped, or D-shaped with parts for attaching the two ends of the device to form a ring-like structure. In certain embodiments, the parts for attaching include a female and male end. Embodiments of the device may also be designed like a rivet with two separate and male and female pieces which can be fastened together. In order to minimize the tearing of mucosa by the fastening device, certain embodiments do not include a round cross-sectional area. Instead, the cross-section of certain embodiments of the device is flattened, oval, polygonal, rectangular, or square. In particular embodiments, the cross-section of the part of the device penetrating the tissue is flattened, oval, polygonal, rectangular, or square. Such a cross-sectional shape reduces the likelihood of the device from tearing through the tissue which has been closed. The device may be one piece molded from a biocompatible, bioresorbable polymeric material.

Embodiments of the fastening devices range in size depending upon the particular use of the device and the size of the patient. For example, infants or children may require smaller sized devices than adults. In certain embodiments, the fastening devices are approximately 0.2 cm to approximately 2 cm in length in length and width. In certain embodiments the device is approximately 0.5 cm to approximately 1.0 cm in length and width. In certain embodiments the device is approximately 0.5 cm to approximately 1.5 cm in length and width. In certain embodiments the device is approximately 0.75 cm to approximately 1.5 cm in length and width. In certain embodiments the device is approximately 1 cm to approximately 2 cm in length and width. In certain embodiments, the devices are approximately square. In certain embodiments, the devices are longer in length than in width. For example, the device may be approximately 0.5-2 cm in length and approximately 0.2-1.5 cm in width. In certain embodiments, the device is approximately 0.5-1.5 cm in length and 0.2-1.0 cm in width. In certain embodiments, the ratio of length to width ranges from approximately 1.5:1 to approximately 10:1. In certain embodiments, the ratio of length to width ranges from approximately 1.5:1 to approximately 5:1. In certain embodiments, the ratio of length to width is approximately 1.5:1, approximately 2:1, approximately 2.5:1, approximately 3:1, approximately 3.5:1, approximately 4:1, approximately 4.5:1, or approximately 5:1. These dimensions represent the dimensions of the fastening device in the open or closed configuration. These dimensions also represent mere examples. Embodiments of the invention also encompasses larger and smaller fastening devices.

The thickness of the device may also determined by the use. The thickness of the device can be important in reducing or preventing the tearing of tissue by the device. Thicker devices are typically less prone to tear tissue held by the device. In certain embodiments, the thickness of the device is typically approximately 0.2 mm to approximately 2 mm. In certain embodiments, the thickness or height is approximately 0.5 mm to approximately 2 mm. In certain embodiments, the height is approximately 0.5 mm to approximately 1.5 mm. In certain embodiments, the height is approximately 0.5 mm, approximately 0.6 mm, approximately 0.7 mm, approximately 0.8 mm, approximately 0.9 mm, approximately 1.0 mm, approximately 1.1 mm, approximately 1.2 mm, approximately 1.3 mm, approximately 1.4 mm, approximately 1.5 mm, approximately 1.6 mm, approximately 1.7 mm, approximately 1.8 mm, approximately 1.9 mm, or approximately 2.0 mm. Devices thicker or thinner than the above recited ranges are also considered to be part of the present invention.

In certain embodiments the fastening device includes parts for fastening the device in the closed position. In certain embodiments, these fastening parts are located on the ends of the devices, for example, on the end of a member of the device. Several different configurations of fastening mechanisms may be used. In certain embodiments, the fastening parts include male and female ends for fastening closed the device. The male end may be pointed or sharp in order to pierce mucosal tissue. The male end may also include a swelling, flange, ridge, or other feature to aid in fastening closed the device. The female end may include a ring, hole, or other orifice for receiving the male end. In certain embodiments, the fastening step is reversible. In other embodiments, the fastening step is irreversible. When the fastening is irreversible, the device may be cut with scissors or other sharp instrument to remove it, or the device may be made of a bioresorbable material and simply degrade over time. In certain embodiments, the instrument used to place the fastening device is designed to allow the treating physician to easily fasten the device. In certain embodiments, the device is fastened using a specially designed instrument as described herein. In other embodiments, the device is fastened using standard surgical instruments such as forceps or clamps to crimp the device into place.

The fastening device may be constructed of any biocompatible material. In certain embodiments, the material is rigid enough to allow an end of the device (e.g., the male end) to pierce mucosa. The material may be a natural or non-natural material. The material may be bioresorbable or non-bioresorbable. The material may be polymeric. In certain embodiments, the fastening device is made of a bioresorbable polymeric material. In certain embodiments, the fastening device is made of a bioresorbable, synthetic polymeric material. In certain embodiments, the polymer is a co-polymer. In certain embodiments, the polymer is block polymer. In certain embodiments, the polymer is linear polymer. In certain other embodiments, the polymer is a branched polymer. In certain embodiments, the polymer is a dendritic polymer. In certain embodiments, the polymer is a cross-linked polymer. In certain embodiments, the polymer is a polyester, polyurethane, polyvinyl chloride, polyethylene, polyolefin, polyanhydride, polyamide, polycarbonates, polycarbamate, polyacrylate, polymethacrylate, polystyrene, polyurea, polyether, polyalkylether, or polyamine. Exemplary polymers that may be used to make the device include poly(lactic acid), poly(glycolic acid), poly(lactic-co-glycolic acid) (PLGA), poly(anhydride), polyphosphazenes, and poly(caprolactone). In certain embodiments, the polymer is a poly(glycolide-co-lactide) (PLGA). In certain embodiments, the device is made of 50% D,L-lactide and 50% glycolide co-polymer. In certain embodiments, the device is made of 50% L-lactide and 50% glycolide co-polymer. In certain embodiments, the device is made of 85% D,L-lactide and 15% glycolide co-polymer. In certain embodiments, the device is made of 85% L-lactide and 15% glycolide co-polymer. In certain embodiments, the device is made of 90% D,L-lactide and 10% glycolide co-polymer. In certain embodiments, the device is made of 90% L-lactide and 10% glycolide co-polymer. In certain embodiments, the polymer is polyglycolic acid. In certain embodiments, the polymer is poly-β-hydroxybutyrate. In certain embodiments, the polymer is polyacrylic acid ester. In certain embodiments, the device is made of Pebax, Polyimide, Braided Polyimide, Nylon, PVC, Hytrel, HDPE, or PEEK. In certain embodiments, the device is made of a fluorinated polymer such as PTFE, PFA, FEP, and EPTFE. In certain embodiments, the device is made of latex. In certain embodiments, the device is made of a natural polymer. In certain embodiments, the natural polymer is a polysaccharide such as cellulose or derivatives thereof. In certain embodiments, the natural polymer is a protein. The fastening device may be made of a material that is bioabsorbed after the device is no longer needed (e.g., after the tissues have healed). For example, the device may degrade in vivo after 1 week, 2 weeks, 3 weeks, 1 month, 2 months, 3 months, 4 months, 5 months, 6 months, etc. In certain embodiments, the device is designed to degrade after approximately 4-6 weeks in vivo.

In certain embodiments, the fastening device may also be constructed of a shape memory plastic or metallic material. Such shape memory plastic or metallic materials can return to some previously defined shape or size when subjected to the appropriate thermal procedure or similar form of energy. That is, shape memory alloys or plastics can be plastically deformed at some relatively low temperature and, upon exposure to some higher temperature, will return to their original shape. Metal materials that exhibit the shape memory effect include a number of copper allow systems and the alloys of gold-cadmium, nickel-aluminum, and iron-platinum. In addition, plastic materials that exhibit the shape memory effect have been developed and include, for example, materials disclosed in U.S. Pat. Nos. 6,720,402, 6,388,043, 6,160,084 (each included by reference herein). In certain embodiments, the fastening device may be provided in a shape that is easily inserted into the mucosa. Thermal energy may then be applied to cause the fastening device to change shape (e.g., make the fastening device close together), thus providing a rapid closure of the fastener through the mucosal flaps with merely an application of heat or energy.

In certain embodiments, the fastening device may comprise an elastomeric material, including for example, a polyhydroxyalkanoate (PHA) material. In certain embodiments, the fastening device may comprise a crystalline or amorphous polymer combined with an elastomeric polymer. For example, a highly crystalline polylactide may be blended with a polyhydroxybutarate; specifically 80-97% Poly-L-Lactides (PLLA) and 20-3% PHA in certain embodiments. Similarly, caprolactone or trimethyl carbonate may be added to a crystalline polymer to make it more elastic. Elasticity of the construct can be achieved through the addition of the caprolactone or trimethyl carbonate to a lactide or glycolide monomer since the caprolactone and trimethyl carbonate have relatively low melting temperatures (e.g., 60° C. for carpolactone).

Such material can provide an elasticity that is similar to that of the surrounding tissue. By providing a similar elasticity to the surrounding material, the fastening device can flex with the surrounding tissue. This can increase the ability of the fastening device to maintain approximation of the tissue during healing, and can reduce the likelihood that the fastening device will tear or otherwise damage the surrounding tissue.

In certain embodiments, the fastening device is coated. The coating may be biocompatible, and in certain embodiments, the coating is a polymeric coating. The coating may provide the release of a pharmaceutical agent. The agent may be released over hours, to days, to weeks, to months. In certain embodiments, the coating is a polymeric coating impregnated with a therapeutic agent. Classes of therapeutic agents that may be delivered by the device include DNA, RNA, nucleic acids, proteins, peptides, or small molecules. Exemplary therapeutic agents include antibiotics, anti-inflammatory agents, corticosteroids, vasoconstrictors, vasodilators, coagulants, pain relievers, etc. In other embodiments, the coating is Teflon. The device may be coated with a polysaccharide such as hyaluronate. The coating may also include a radioopaque agent for imaging of the device. The coating may also be designed to make the fastening device more biocompatible.

In certain embodiments, the invention also provides an instrument for fastening or closing tissue using the inventive fastening devices. In certain embodiments, the instrument is designed for use in the oronasopharynx. In certain particular embodiments, the instrument is designed for use in a particular procedure or surgery. In certain embodiments, the instrument is designed for use in spaces of the head and neck. For example, an instrument may be designed for closing the tonsillar fossa in a tonsillectomy or UPPP with or without tonsillectomy. In certain embodiments, the instrument is designed for use in a UPF procedure. In certain particular embodiments, the instrument is designed for use in closing a pharyngeal flap. In certain embodiments, the instrument is designed for closing a laryngeal or pharyngeal defect. In certain embodiments, the instrument is designed for use in tumor removal. In certain embodiments, the instrument is designed for use in a dental procedure. The instrument may include a long neck for reaching spaces in the oronasopharynx. The instrument may also be relatively thin and/or compact for operating in such confined spaces. The instrument may include a comfortable handle with a triggering mechanism for fastening the inventive fastening device around approximated tissue(s). The instrument may include a plurality of fastening devices. For example, the instrument may include at least 5, at least 10, at least 15, at least 20, or at least 25 of the fastening devices. In certain embodiments, the fastening devices are provided in a cassette for easy re-loading of the instrument. In certain embodiments, the instrument includes a mechanism for automatically engaging a new fastening device after one has been placed. In certain embodiments, the instrument includes a mechanism for fastening the fastening devices after tissue has been engaged. In certain embodiments, the instrument is disposable. In other embodiments, the instrument is suitable for sterilization after each use. Therefore, the instrument may be repeatedly used by reloading the instrument with fastening devices or cassettes of fastening devices.

A fastening device may be placed and held at the end of the instrument so it can be maneuvered in tight spaces to the site where it is to be used. The instrument may assist in the grasping of tissue to be approximated or another surgical instrument such as forceps may be used. Once the tissue has been approximated as desired and the fastening device is in place, it is fastened and the fastened device is released with the tissue left held in place by the device. The instrument may automatically engage a new fastening device, or the operator may accomplish this manually. As will be appreciated by one of the skill in the art, various means for fastening the inventive fastening devices using the instrument could also be used in designing the instrument.

The inventive fastening devices may be packaged in kits for convenience. A kit may also include an instrument for using the fastening devices. The kit may also include instructions for using the components of the kit. In certain embodiments, the kits may also include all or some of the following items: an instrument for using the closures, forceps, clamps, pharmaceutical agents (e.g., anesthetics, pain relievers, hemostatic agents, anti-inflammatory agents, antibiotics, vasoconstrictors, etc.), nasal sprays, gauze, disinfectant, and instructions for using the contents of the kit. In certain embodiments, the kits are sterilely package for convenient use by a surgeon or other health care professional.

Embodiments of the inventive system have a wide variety of uses in medicine. In certain embodiments the system is particularly useful for closing in a surgical procedure. The devices may be used on any tissue in the body of any animal. In certain embodiments, the subject is a mammal. In certain particular embodiments, the subject is a human. As discussed, the system is particularly useful for closing or approximating mucosal tissues such as those found in the oral cavity, nasopharynx, hypopharynx, nasal cavity, pharynx, larynx, or esophagus. The system may also be used in the gastrointestinal or genitourinary system. Exemplary procedures that may utilize the inventive devices include UPPP, tonsillectomy, UPF procedure, tumor removal, dental extraction, dental procedure, total or partial laryngectomy, esophagectomy, pharyngectomy, etc. The device may be used to approximate two tissues, passing the device through the tissues, and fastening the device closed. These steps may be aided by the instruments described herein. In certain embodiments, the fastening devices are used with a sealant such as a natural or synthetic sealant (e.g., fibrin-based sealant, collagen-based sealant, fibrin-based sealant).

Certain embodiments comprise a fastening device for fastening tissue comprising a body that forms a partial ring shape. The body may include: an interior surface directed towards the center of the partial ring shape, where the interior surface is substantially flat, and an exterior surface directed away from the center of the partial ring shape. In certain embodiments, the first fastening member is disposed on a first end of the body and a second fastening member is disposed on a second end of the body. In exemplary embodiments, the first fastening member and the second fastening member are each configured to pierce tissue, which may be mucosal tissue.

In certain embodiments, the first fastening member and the second fastening member are configured to be coupled together. The body may comprise a hinge so that the first fastening member can be brought closer to the second fastening member. In certain embodiments, the device is made of a biocompatible, bioresorbable material. The device may be coated with a protein or growth factor-based product to enhance healing of the tissue during use.

Other embodiments comprise a kit including: a plurality of fastening devices of different sizes, and a first instrument for fastening the fastening devices to tissues. Still other embodiments may comprise kits with pharmaceutical agents, a second instrument for removing the fastening devices, and instructions for using the fastening devices, the first instrument, and the second instrument.

Exemplary embodiments comprise a method of fastening tissue, the method comprising: providing a fastening device; approximating a first portion of tissue to a second portion of tissue; piercing the first portion of tissue with the first fastening member; and piercing the second portion of tissue with the second fastening member, wherein the interior surface of the partial ring structure engages the first portion of tissue and the second portion of tissue. Certain embodiments further comprise crimping the body of the fastening device after piercing the first portion of tissue with the first fastening member and the second portion of tissue with the second fastening member. In specific embodiments, the fastening device is included in a cassette of multiple fastening devices. Exemplary embodiments may also comprise utilizing an instrument place the fastening device in a desired location and actuating the instrument to cause a first fastening member to pierce a first portion of the tissue.

In certain embodiments, the steps are performed in the context of a uvulopalatopharyngoplasty (UPPP) with or without tonsillectomy procedure. The steps may be performed in the context of a tonsillectomy procedure. In other embodiments, the steps are performed in the context of a uvulopalatal flap (UPF) procedure.

Exemplary embodiments comprise a method of fastening tissue, the method comprising: providing a fastening device with a first fastening member configured to pierce tissue and a second fastening member configured to couple to the first fastening member; locating the fastening device proximal to a first portion of tissue and a second portion of tissue; piercing the first portion of tissue with the first fastening member; piercing the second portion of tissue with the first fastening member; and coupling the first fastening member to the second fastening member. In certain embodiments, the fastening device comprises a substantially flat surface that engages the first portion of tissue and the second portion of tissue. In certain embodiments, the first fastening member comprises a sharp point and the second fastening member comprises an aperture configured to receive the first fastening member.

Certain embodiments may comprise a fastening device for fastening tissue, where the fastening device comprises: a body that forms a partial ring shape, where the body comprises a first end and a second end; a first fastening member proximal to the first end of the body, wherein the first fastening member comprises an aperture; and a second fastening member proximal to the second end of the body, wherein the second fastening member comprises a plurality of engagement members configured to be inserted through the aperture. In specific embodiments, the plurality of engagement members may be spaced along the body. In particular embodiments, the plurality of engagement members may be configured to restrict the second end from being withdrawn from the aperture when an engagement member is inserted through the aperture.

In certain embodiments, the body may form a ring shape when the an engagement member is inserted through the aperture. In specific embodiments, the plurality of engagement members can be configured to allow the ring shape to be contracted but not expanded when an engagement member is inserted through the aperture. In specific embodiments, the engagement members and the aperture may form a ratcheting mechanism. In certain embodiments, the body may comprise a non-circular cross-section.

Particular embodiments include a method of fastening tissue, where the method may comprise: providing a fastening device according to an exemplary embodiment described herein; approximating a first portion of tissue to a second portion of tissue; piercing the first portion of tissue and the second portion of tissue with the second fastening member; and inserting an engagement member through the aperture of the first fastening member.

Certain embodiments include a fastening device for fastening tissue, where the fastening device may comprise: a first suture comprising a first fastening member; a second suture comprising a second fastening member; and a coupling member coupling the first suture to the second suture, where the coupling member is configured to slide relative to the first and second suture. In specific embodiments, the coupling member may comprise a first aperture and a second aperture, and the first suture may extend through the first aperture and the second suture may extend through the second aperture.

In particular embodiments, the first fastening member may be coupled to a first end of the first suture and the second fastening member may be coupled to a first end of the second suture. In certain embodiments, the first fastening member and the second fastening member may each comprise a pointed end configured to penetrate tissue.

Specific embodiments include a method of fastening tissue, where the method may comprise: providing a fastening device according to an exemplary embodiment described herein; approximating a first portion of tissue to a second portion of tissue; piercing the first portion of tissue with the first fastening member; piercing the second portion of tissue with the second fastening member; and moving the coupling member toward the first and second fastening members. In particular embodiments, the method may comprise: tying a first knot in the first suture so that the coupling member is positioned between the first knot and the first fastening member; and tying a second knot in the second suture so that the coupling member is positioned between the second knot and the second fastening member. In certain embodiments, the method may comprise: tying the first suture and the second suture in a knot so that the coupling member is positioned between the knot and the first and second fastening members.

Particular embodiments include a fastening device for fastening tissue, where the fastening device may comprise: an elongate body with a first end and a second end; a first fastening member proximal to the first end of the elongate body; and a second fastening member proximal to the second end. In specific embodiments, the second fastening member may be configured to move from a first position where the second fastening member is located at a first angle to the elongate body to a second position where the second fastening member is located at a second angle to the elongate body, and where the first angle is greater than the second angle. In particular embodiments, first angle may be approximately 100, 90, 80, 70, or 60 degrees. In certain embodiments, the second angle may be less than 55, 50, 45, 40, or degrees.

Specific embodiments include a method of fastening tissue, where the method may comprise: providing a fastening device according to an exemplary embodiment described herein; approximating a first portion of tissue to a second portion of tissue; placing the second fastening member in the second position described above; inserting the second fastening member through an incision in the first portion of tissue and the second portion of tissue; and placing the second fastening member in the first position described above.

Certain embodiments comprise a fastening device for fastening tissue, where the fastening device may comprise: a first elongate body with a first end and a second end; a first fastening member proximal to the first end of the first elongate body and a penetrating member proximal to the second end of the first body; a second elongate body with a first end and a second end; a second fastening member proximal to the first end of the second elongate body and a receiving member proximal to the second end of the second elongate body. In specific embodiments, the penetrating member and receiving member may be configured for an interference fit.

Particular embodiments include a method of fastening tissue, where the method may comprise: providing a fastening device according to an exemplary embodiment described herein; approximating a first portion of tissue to a second portion of tissue; inserting the penetrating member through the first and second portions of tissue; and coupling the first elongate body and the second elongate body by inserting the penetrating member into the receiving member.

Certain embodiments comprise a fastening device for fastening tissue, where the fastening device may comprise: an elongate body comprising a central portion, a first end and a second end; a first penetrating member proximal to the first end; a second penetrating member proximal to the second end; a first plurality of engagement members coupled to the elongate body between the central portion and the first end, where the first plurality of engagement members are angled away from the first end; and a second plurality of engagement members coupled to the elongate body between the central portion and the second end, where the second plurality of engagement members are angled away from the second end. In particular embodiments, the elongate body may comprise a first side and a second side, and the first plurality of engagement members may be disposed on the first side and the second side of the elongate body. In certain embodiments, the second side may be opposing the first side.

Specific embodiments include a method of fastening tissue, where the method may comprise: providing a fastening device according to an exemplary embodiment described herein; approximating a first portion of tissue to a second portion of tissue; inserting the first penetrating member into the first portion of tissue; and inserting the second penetrating member into the second portion of tissue. In particular embodiments, the fastening device may comprise an elastomeric material. In certain embodiments, the elastomeric material may be a polyhydroxyalkanoate material. In specific embodiments, the elastomeric material may be combined with a crystalline or amorphous polymer. In particular embodiments, the fastening device may have an elasticity equivalent to that of mucosal tissue. In certain embodiments, the fastening device may comprise a shape memory material.

### BRIEF DESCRIPTION OF THE DRAWINGS

FIG. 1 illustrates a perspective view of a fastening device according to exemplary embodiments of the present disclosure.

FIG. 2 illustrates a front view of the embodiment of FIG. 1.

FIG. 3 illustrates a top view of the embodiment of FIG. 1.

FIG. 4 illustrates a bottom view of the embodiment of FIG. 1.

FIG. 5 illustrates a side view of the embodiment of FIG. 1.

FIG. 6 illustrates a partial cross-section view of the embodiment of FIG. 1 during use.

FIG. 7 illustrates a perspective view of a fastening device according to exemplary embodiments of the present disclosure.

FIG. 8 illustrates a front view of the embodiment of FIG. 1.

FIG. 9 illustrates a top view of the embodiment of FIG. 1.

FIG. 10 illustrates a bottom view of the embodiment of FIG. 1.

FIG. 11 illustrates a side view of the embodiment of FIG. 1.

FIG. 12A illustrates a perspective view of a fastening device according to exemplary embodiments of the present disclosure.

FIG. 12B illustrates a side view of the embodiment of FIG. 12A.

FIG. 12C illustrates an end view of the embodiment of FIG. 12A.

FIG. 13 illustrates a perspective view of a fastening device according to exemplary embodiments of the present disclosure.

FIG. 14 illustrates a perspective view of a fastening device according to exemplary embodiments of the present disclosure.

FIG. 15 illustrates an instrument configured to place a fastening device according to exemplary embodiments of the present disclosure.

FIGS. 16A-16C illustrate a surgical procedure utilizing fastening devices according to exemplary embodiments of the present disclosure.

FIGS. 17A-17B illustrate a surgical procedure utilizing fastening devices according to exemplary embodiments of the present disclosure.

FIG. 18 illustrates a perspective view of a fastening device according to exemplary embodiments of the present disclosure.

FIG. 19 illustrates a perspective view of a fastening device according to exemplary embodiments of the present disclosure.

FIG. 20A illustrates a perspective view of a fastening device in a first position, according to exemplary embodiments of the present disclosure.

FIG. 20B illustrates a perspective view of the fastening device of FIG. 20A in a second position.

FIG. 21 illustrates a perspective view of the fastening device of FIG. 20A in an installed position.

FIG. 22 illustrates a perspective view of a fastening device according to exemplary embodiments of the present disclosure.

FIG. 23 illustrates a perspective view of a fastening device according to exemplary embodiments of the present disclosure.

### DETAILED DESCRIPTION OF EXEMPLARY EMBODIMENTS

Embodiments of the present disclosure provide a system and method for closing or approximating tissues, particularly mucosal tissues. The system can be particularly useful in surgeries and procedures involving the mucosal surfaces of the head and neck such as the oronasopharynx. Traditionally, placing sutures in mucosal tissues has been difficult given the propensity of the sutures to tear through the mucosal tissue. This is particularly problematic when tension is placed on the suture. The placement of sutures in the oronasopharynx, especially for procedures such as UPPP, UPF, or tonsillectomy, is especially time-consuming and difficult given the propensity of mucosa to tear and space constraints in this area. Embodiments of the present invention provide a specially designed fastening device for use in mucosal tissues that is useful in surgeries of the head and neck. Embodiments of the present invention also provide instruments for placing the fastening devices, kits including the devices and/or the instruments, and methods of using the novel fastening devices and/or the instruments.

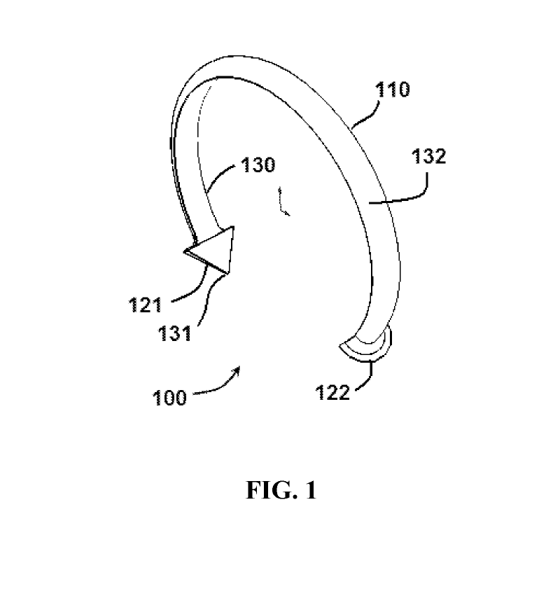

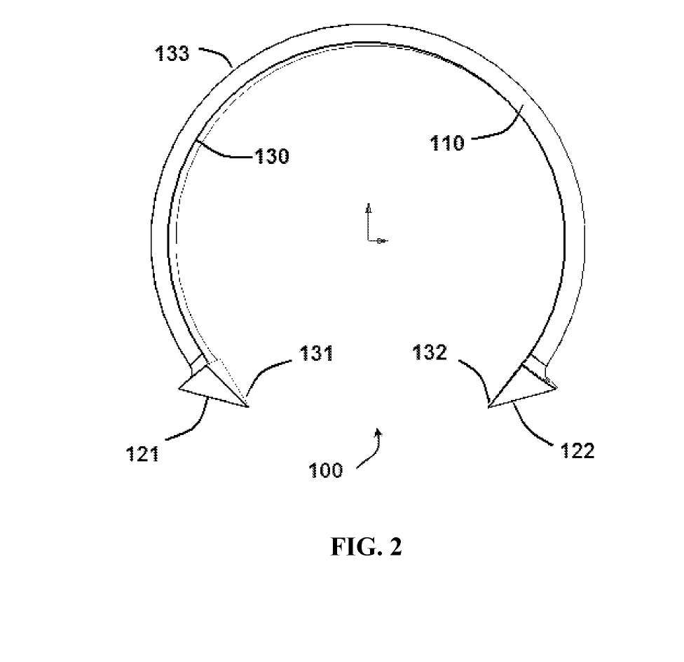

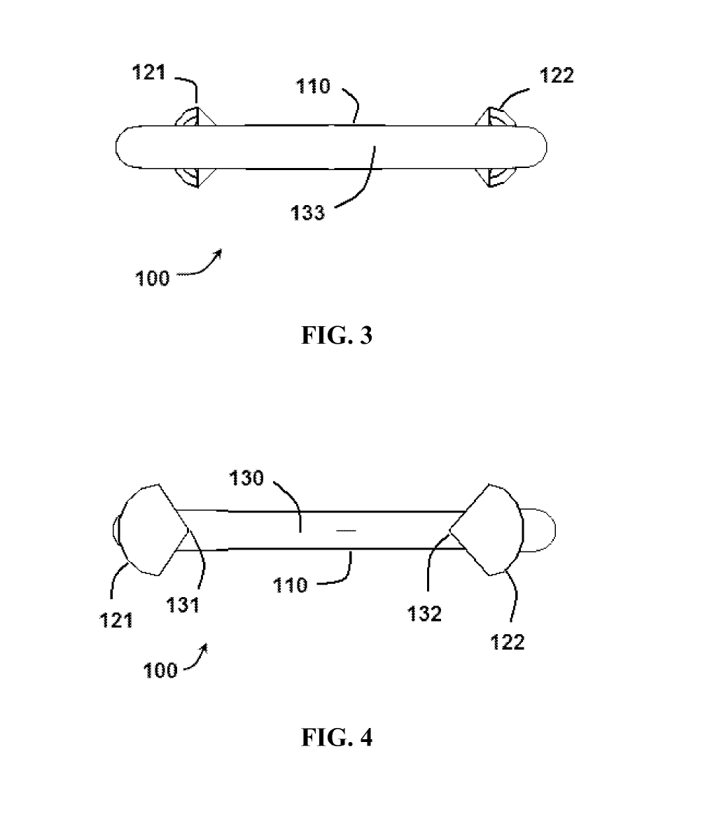

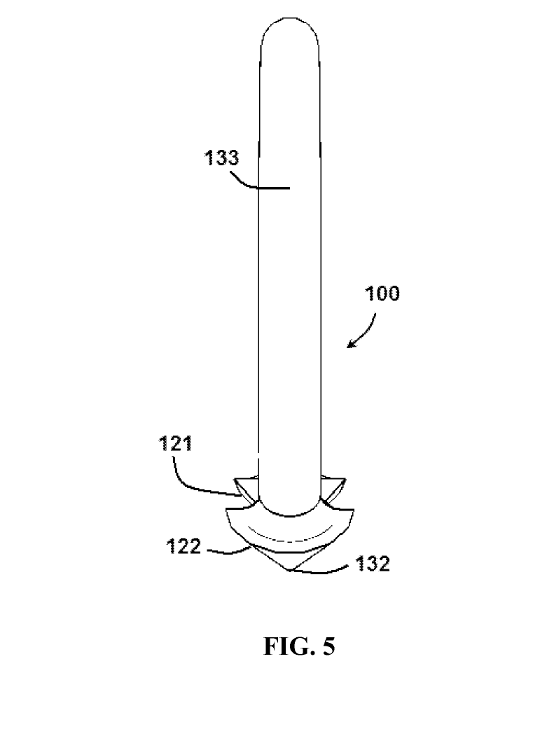

Referring now to FIGS. 1-5, an exemplary embodiment of a fastening device 100 comprises a body 110 with fastening members 121 and 122. In the embodiment shown in FIGS. 1-5, body 110 is a segment of a circle. In other embodiments, body 110 may comprise other curved shapes or segments angled with respect to each other to form, for example, an open polygon shape. In exemplary embodiments, body 110 forms a partial ring shape. In certain embodiments, fastening members 121 and 122 are arrow-shaped and contain a sharp point 131 132, respectively, configured to pierce mucosa or other tissue. Other embodiments may comprise fastening members of different shapes or configurations. For example, certain embodiments may contain fastening members that may be coupled together. Specific embodiments may comprise male-female snap connectors.

In certain embodiments, body member 110 comprises an interior surface 130 directed towards the center of fastening device 110 and an exterior surface 133 directed away from the center of fastening device 110. In exemplary embodiments, interior surface 130 is substantially flat, and can engage the mucosa (or other tissue being closed) during use. During use, the engagement of interior surface 130 with the mucosa spreads the forces exerted from fastening device 110 to the mucosa over the interior surface 130. Such spreading of the forces reduces the pressure exerted on the mucosa as compared to a fastening device with a non-flat mucosa-engaging surface. Fastening device 100 may therefore reduce the likelihood that the mucosa or other tissue being closed will be torn or ruptured.

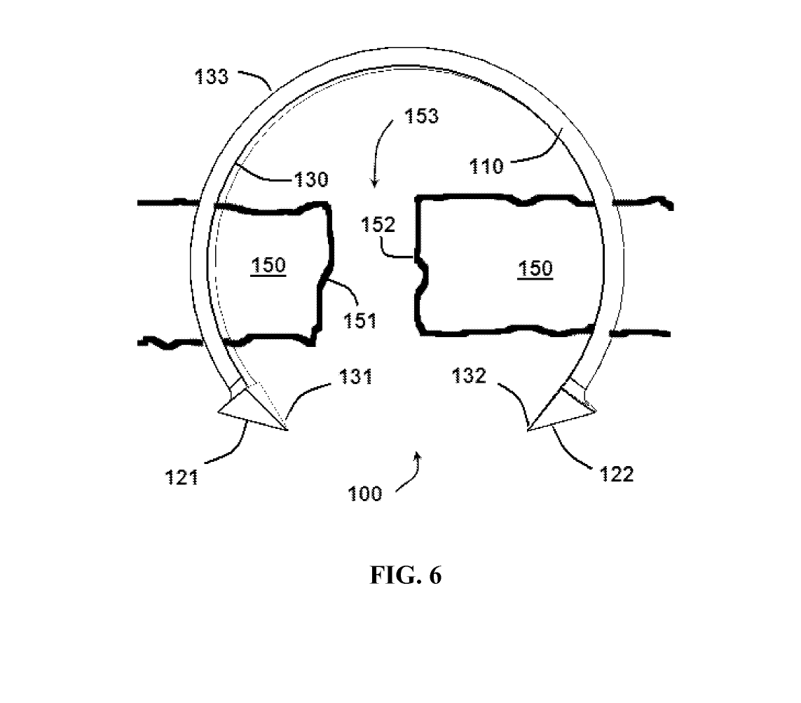

Referring now to FIG. 6, during use fastening member 121 may pierce one side 151 of an opening 153 (for example, a cut, tear, hole, etc.) in tissue 150 while fastening member 122 may pierce the other side 152 of opening 153 in tissue 150. Fastening device 100 can therefore be used to bring each side 151, 152 closer together to assist in closing opening 153 and expediting healing of tissue 150.

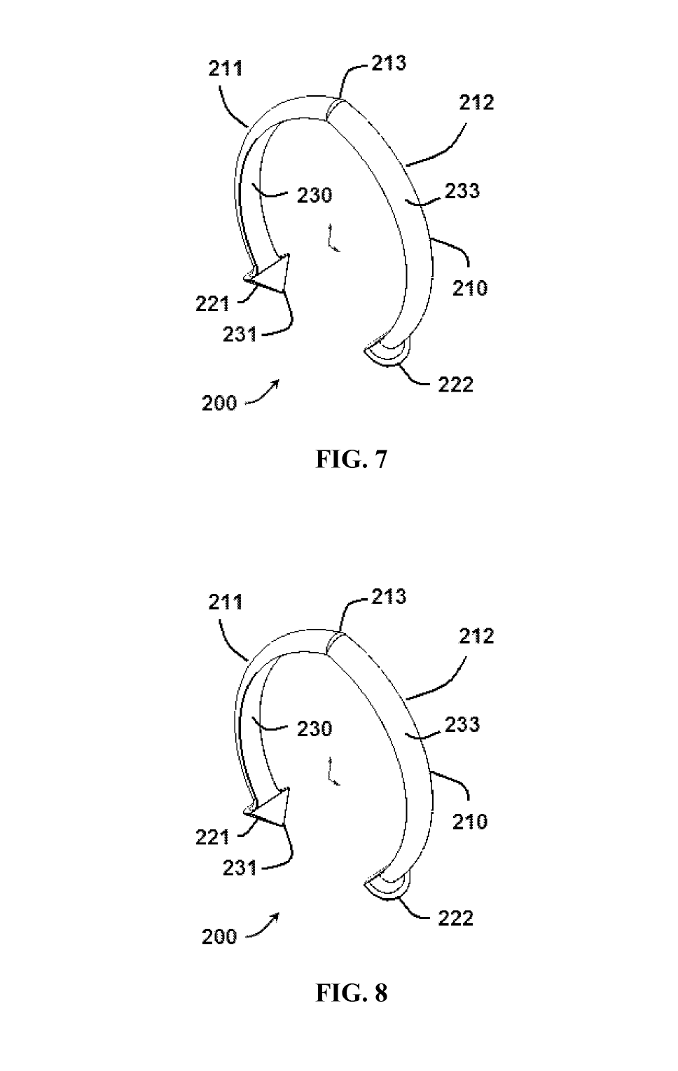

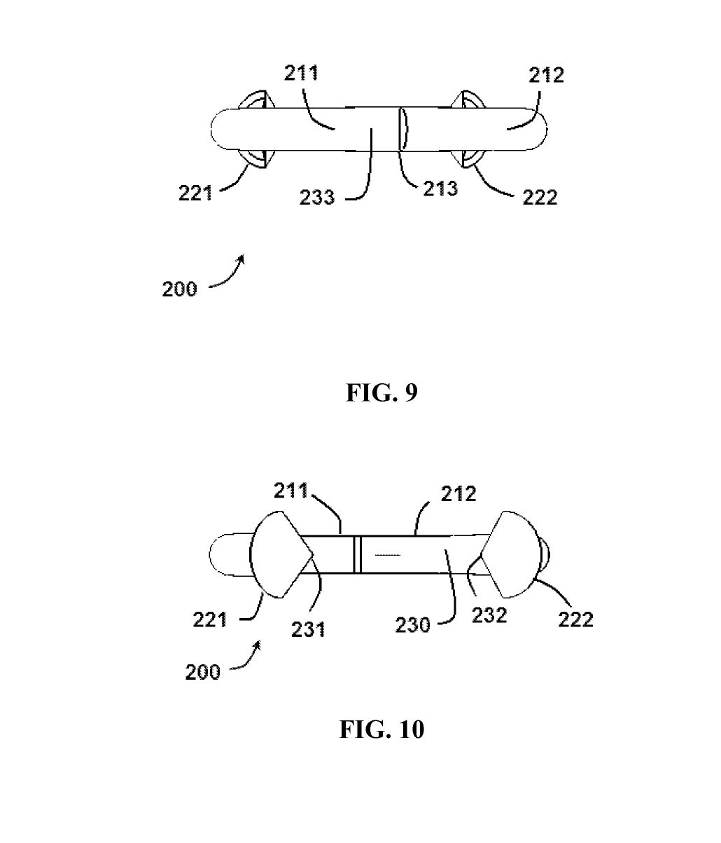

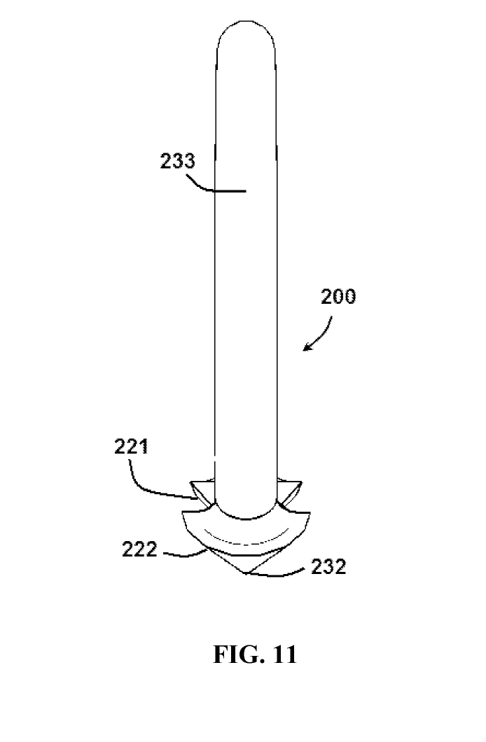

Referring now to FIGS. 7-11, an exemplary embodiment of a fastening device 200 comprises a body 210 with fastening members 221 and 222 with points 231, 232 respectively. The embodiment shown in FIGS. 7-11 is similar to that shown in FIGS. 1-6, but comprises a body that is shaped differently. The embodiment shown in FIGS. 6-10, body 210 is formed of two arc-shaped segments 211, 212 that meet at a hinge 213. In specific embodiments, hinge 213 may be a “living hinge” formed by a thin section of material that allows segments 211, 212 to flex toward or away from each other. In certain embodiments, body 210 is configured so that segments 211 and 212 may be forced towards each other to provide a crimping-type action. In certain embodiments, body 210 may be plastically deformed at hinge 213 so that fastening members 221 and 222 are brought closer together. Fastening members 221 and 222 may also be used to pierce tissue on each side of an opening, and therefore provide a closing force to the opening. Similar to the previously described embodiment, body 210 comprises a flat interior surface 230 that engages the mucosa or tissue during use and an exterior surface 233.

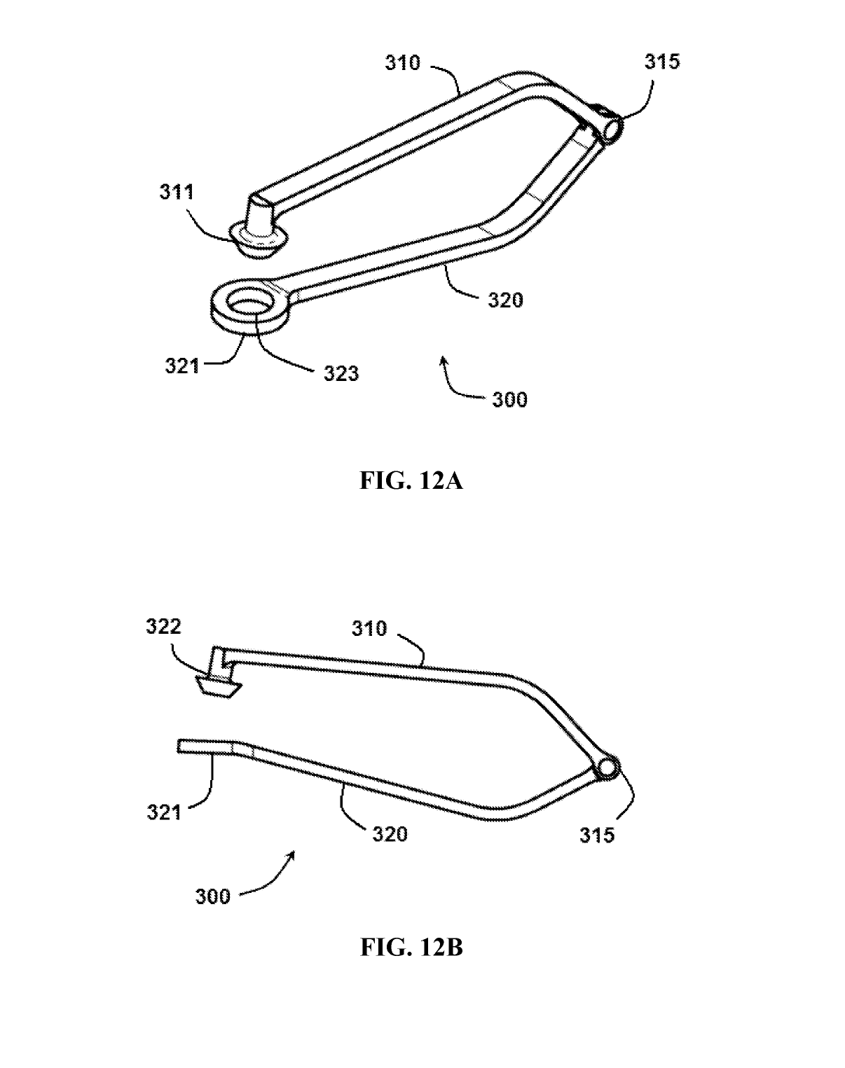

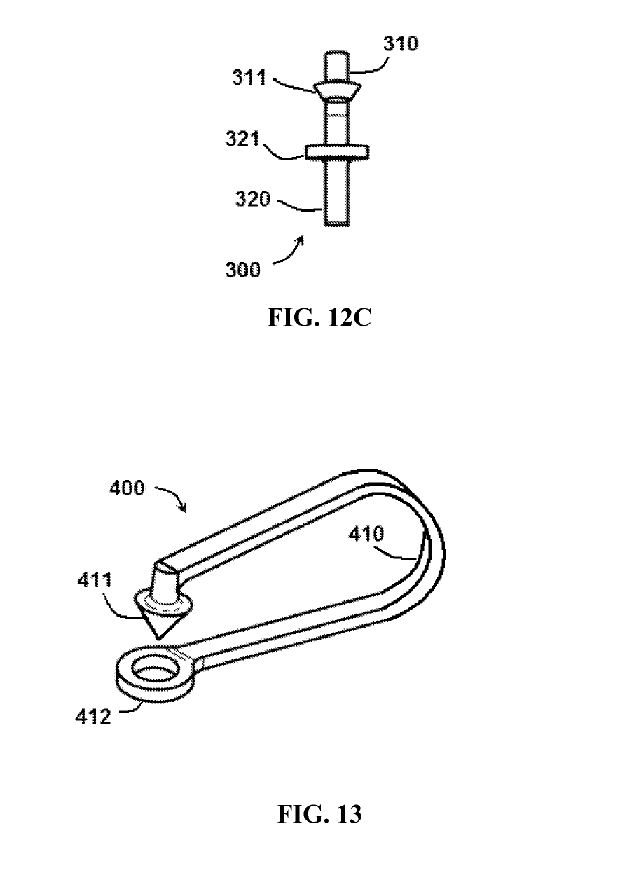

Referring now to FIG. 12A-12C, an exemplary embodiment of a fastening device 300 comprises a first member 310 coupled to a second member 320 at a hinge 315. In certain embodiments, hinge 315 may comprise a post and hole design, while in other embodiments hinge 315 may comprise a living hinge design. First member 310 comprises a fastening member 311 at the end opposite of hinge 315. Second member 320 comprises a fastening member 321 at the end of coupling member 320 that is opposite of hinge 315. In certain embodiments, fastening member 321 comprises an aperture 323 and is configured to receive fastening member 311 so that the fastening members are coupled together. Similar to previously-described embodiments, fastening members 311, 321 may be placed on each side of a tissue opening to assist closing and healing the opening. First and second members 310 and 320 may also comprise cross-sections with flat surfaces that engage the tissue.

Referring now to FIG. 13, an exemplary embodiment of a fastening device 400 comprises a curved member 410 with fastening members 411, 412 disposed at the ends of curved member 410. Fastening device 400 operates similar to fastening device 300, but allows curved member to further bend (rather than pivot at a hinge) so that fastening members 411 and 412 are engaged.

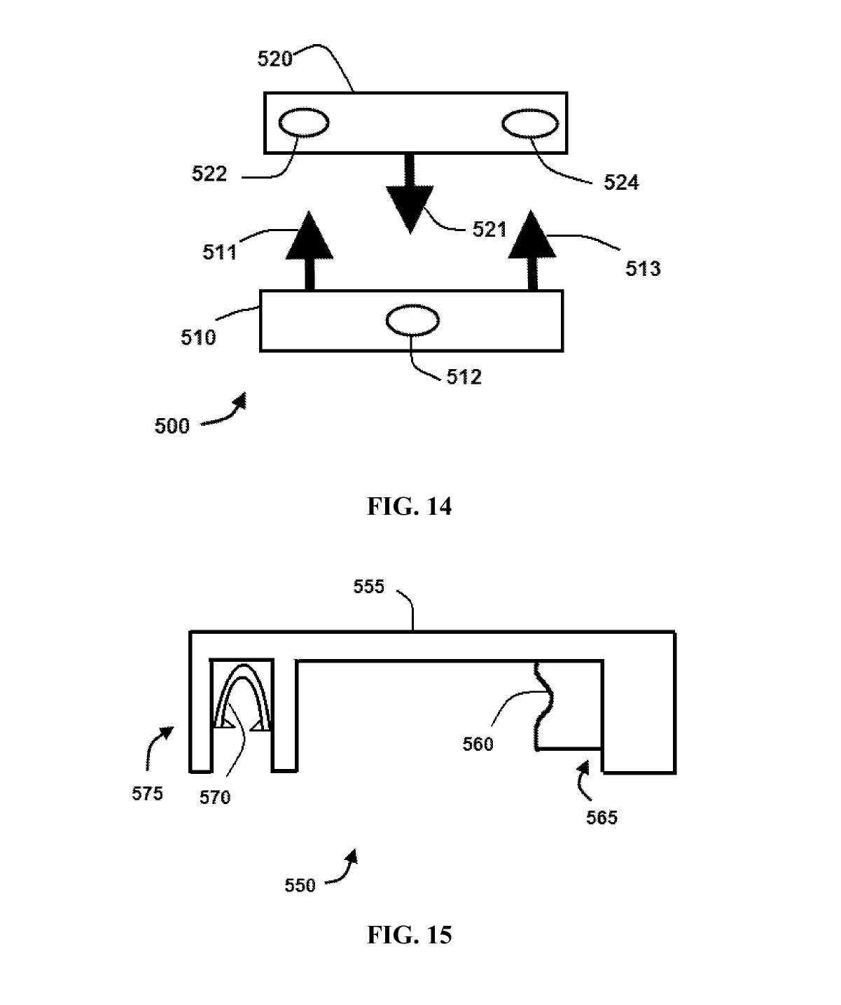

Referring now to FIG. 14, an exemplary embodiment of a fastening member 500 comprises a first member 510 and a second member 520 that can be separated from each other. First member 510 may comprise engagement members 511 and 513 that are configured to pierce tissue, while second member 520 comprises engagement member 521 configured to pierce tissue. First member 510 also comprises an engagement member 512 configured to receive engagement member 521, while second member 520 comprises engagement members 522 and 524 configured to receive engagement members 511 and 513, respectively. In exemplary embodiments, first member 510 may be placed on one side of a tissue opening (not shown) and second member 520 may be placed on an opposing side of the tissue opening. Engagement members 511, 513, and 521 may be used to pierce the tissue and engage engagement members 522, 524, and 512 respectively to fasten together the tissue on each side of the tissue opening. Other embodiments may comprise a different number of engagement members on the first and second members.

Referring now to FIG. 15, an exemplary embodiment of an instrument 550 comprises an elongate portion 555 between a dispensing portion 575 and an actuating portion 565. During use, instrument 550 can be utilized to place a fastening device 570 in a desired location. For example, fastening device 570 may be placed near a tissue opening (not shown) so that fastening device 570 can fasten tissue on each side of the opening. Actuating portion 565 may be actuated (for example, by pulling a trigger or actuator 560) so that dispensing portion 575 can then dispense fastening device 570. In exemplary embodiments, fastening device may be configured similar to any of the previously-described fastening devices in this disclosure. In exemplary embodiments, instrument 550 may comprise a cartridge of fastening devices 570 so that multiple fastening devices 570 can be dispensed in a single procedure.

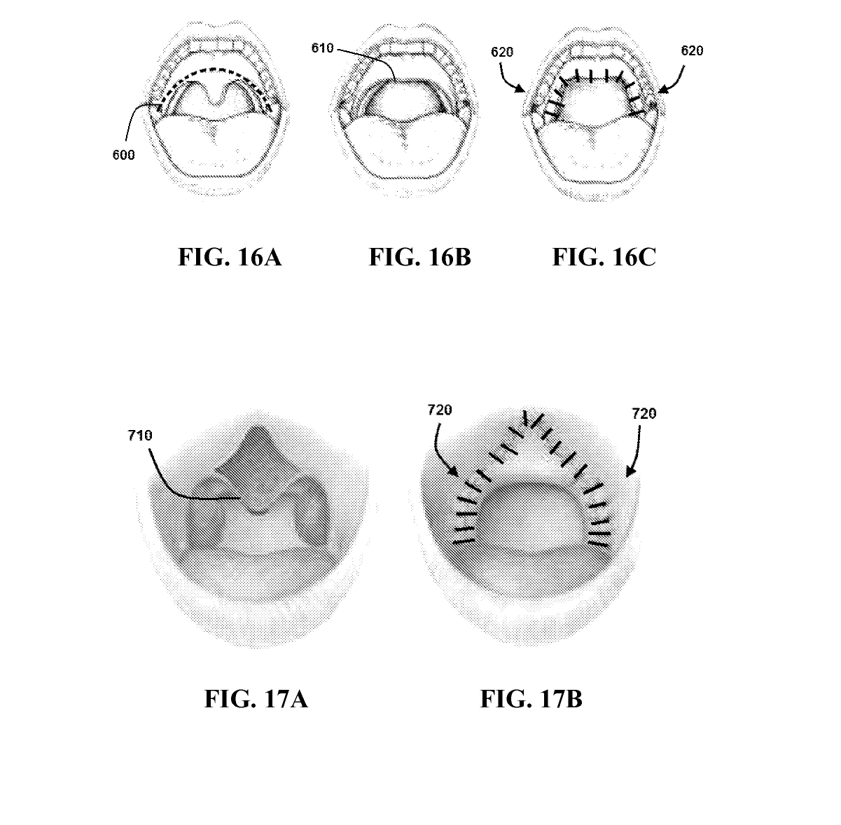

FIGS. 16A-17C show a UPPP/tonsillectomy performed to teat sleep apnea in a patient. This uvulopalatopharyngoplasty (UPPP)/tonsillectomy includes creating an incision at line 600 to remove unwanted tissue. After the incision is made, a tissue opening is formed along line 610. The tissue on each side of the tissue opening can be fastened together using fastening devices 620. In exemplary embodiments, fastening devices 620 may be configured as any one of the previously-described fastening devices in this disclosure. UPPP/tonsillectomy is just one of the many surgical procedures that may be aided by the use of the inventive fastening devices and instruments.

FIGS. 17A-17B illustrate the uvulopalatal (UPF) technique, which is an alternative to UPPP for treating sleep apnea. The UPF technique reduces the risk of velopharyngeal incompetence (VPI), in which the soft palate can not close off the oropharynx from the nasal cavity. The UPF procedure is another procedure that can be aided by the use of the inventive fastening devices. As shown in FIGS. 17A-17B the uvulopalatal flap 710 has been retracted and held in place by fastening devices 720. In exemplary embodiments, fastening devices 720 may be configured as any one of the previously-described fastening devices in this disclosure.

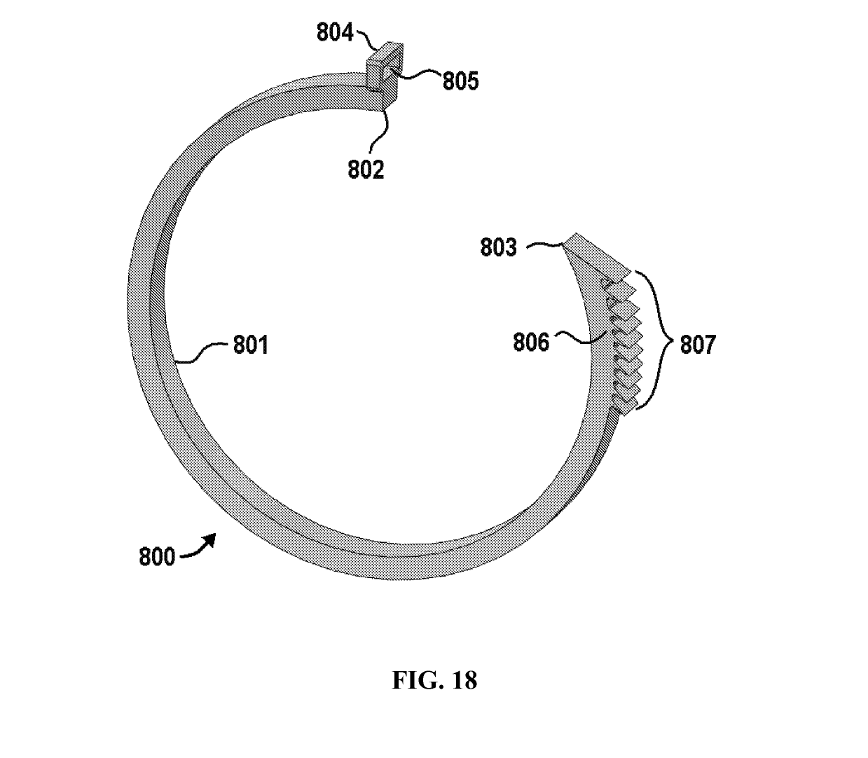

Referring now to FIG. 18, an exemplary embodiment of a fastening device 800 comprises of a body 801 having a first end 802 and a second end 803. Fastening device 800 further comprises a first fastening member 804 having an aperture 805 proximal to first end 802. In the embodiment shown, fastening device 800 comprises a second fastening member 806 proximal to second end 803. Second fastening member 806 may also comprise a plurality of engagement members 807. In the embodiment shown in FIG. 18, body 801 comprises a square cross-section. In other embodiments (not shown), body 801 may comprise a circular cross-section. In the position shown in FIG. 18, body 801 forms a partial ring shape. As described below, fastening device 800 can be manipulated so that body 800 forms a complete ring.

During use, fastening device 800 can be positioned so that tissue (not shown) is positioned between first end 802 and second end 803. Second fastening member 806 can then be directed through the tissue (e.g., by piercing the tissue) towards first end 802 and aperture 805. Second end 803 can then be inserted through aperture 805 until one or more engagement members 807 are inserted through aperture 805. In a specific embodiments, fastening member 806 may pierce tissue on one side of an opening (e.g., a cut, tear, hole, etc.) and rotated through to pierce tissue on the other side of the opening.

Engagement members 807 are configured to restrict second end 803 from being withdrawn from aperture 805 when an engagement member 807 is inserted through aperture 805. In the specific embodiment shown, engagement members 807 are spaced along body 801 and are configured so that they angle away from second end 803. When an engagement member 807 is inserted through aperture 805, second end 803 can be further inserted into aperture 805 so that the ring shape of body 801 to be further contracted. However, when an engagement member 807 is inserted through aperture 805, second end 803 cannot be withdrawn from aperture 805. Aperture 805 and engagement members 807 form a ratcheting mechanism that allows body 801 to be tightened around tissue that is being fastened. This allows a user to adjust body 801 to the desired size and prevents body 801 from opening up and losing tension after the desired size has been set. Such a configuration can bring the tissue closer together to assist in closing the tissue opening and expediting healing of the tissue.

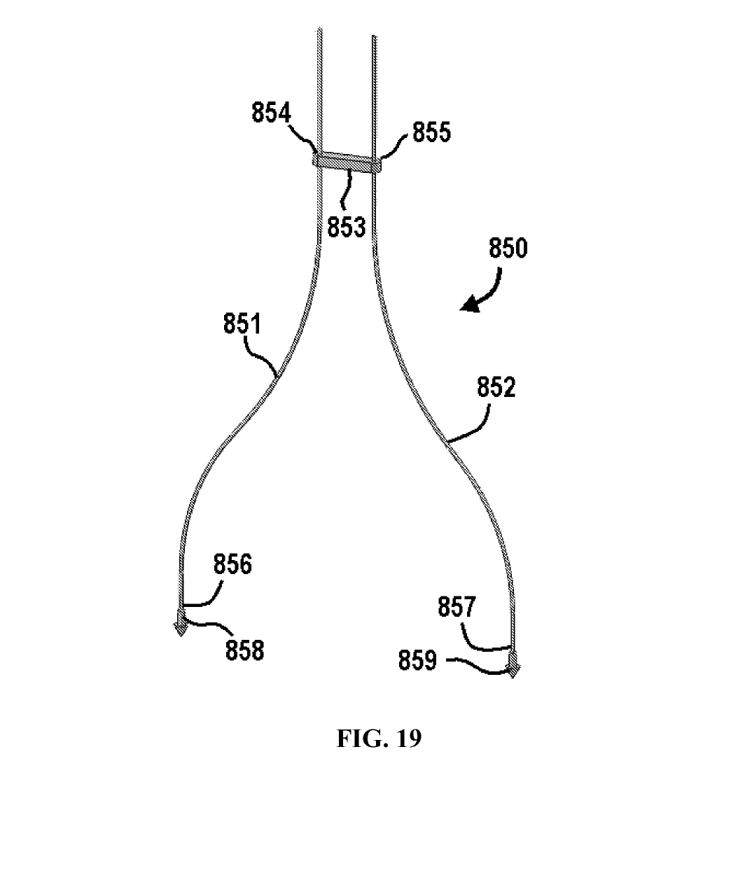

Referring now to FIG. 19, an exemplary embodiment of a fastening device 850 comprises of a first suture 851 and a second suture 852 coupled by a coupling member 853. First suture 851 comprises a first end 856 and second suture 852 comprises a first end 857. In this embodiment, a first fastening member 858 is coupled to first end 856 and a second fastening member 859 is coupled to first end 857. In the embodiment shown, first and second fastening members 858 and 859 each comprise a sharp pointed end to assist in penetrating tissue.

In the illustrated embodiment, coupling member 853 comprises a first aperture 854 and a second aperture 855. First suture 851 extends through first aperture 853 and second suture 852 extends through second aperture 854. During use, fastening member 858 can be used to pierce one side of an opening in tissue while the fastening member 859 can be used to pierce the opposite side of the opening in the tissue. Once fastening members 858 and 859 are anchored in opposite ends of the tissue opening, the coupling member 853 can be drawn toward fastening members 858 and 859. This can bring the tissue closer together to assist in closing the opening and expediting healing of the tissue. In certain embodiments, a knot (not shown) can be tied in each suture 851, 852 so that coupling member 853 is positioned between the knots and the fastening members 858, 859. In still other embodiments, first and second sutures 851, 852 can be tied together in a knot (not shown) so that coupling member 853 is positioned between the knot and the first and second fastening members 858, 859.

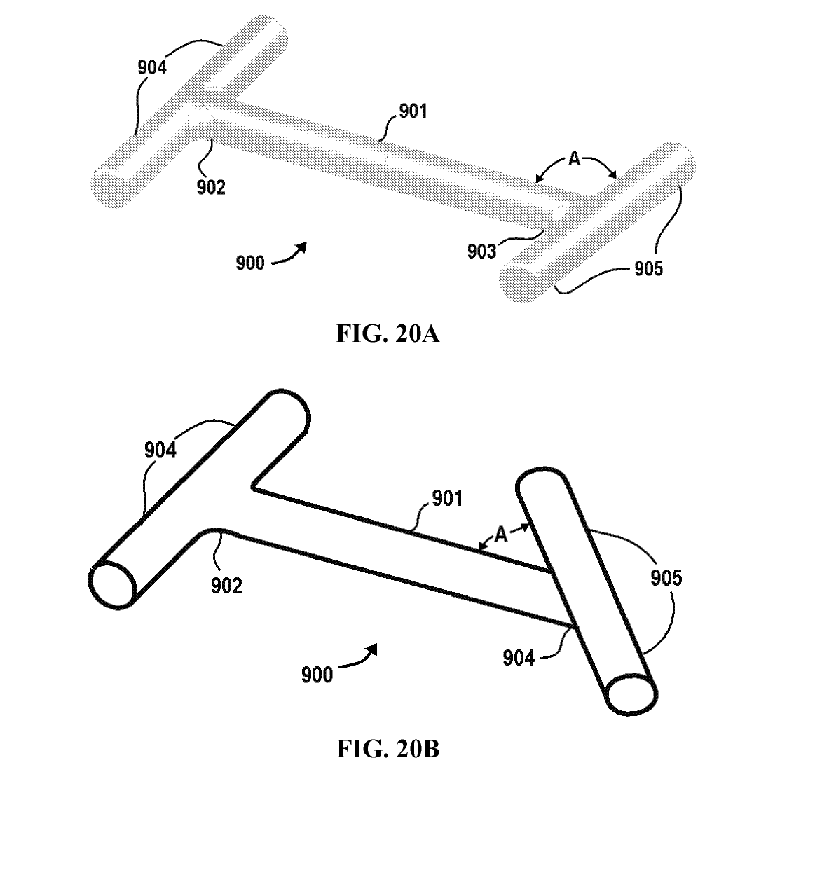

Referring now to FIG. 20A, an exemplary embodiment of a fastening device 900 comprises a body 901 with a first end 902 and a second end 903. A first fastening member 904 is coupled to first end 902 and a second fastening member 905 is coupled to second end 903. In the embodiment shown, second fastening member 905 is capable of moving (e.g., pivoting or swiveling) from a first position shown in FIG. 20A to a second position shown in FIG. 20B. In the first position shown in FIG. 20A, fastening member 905 is located at an angle “A” that is approximately 90 degrees (i.e., perpendicular) to body 901. In the configuration shown in FIG. 20A, body 901 and fastening members 904, 905 form an “H”-shape.

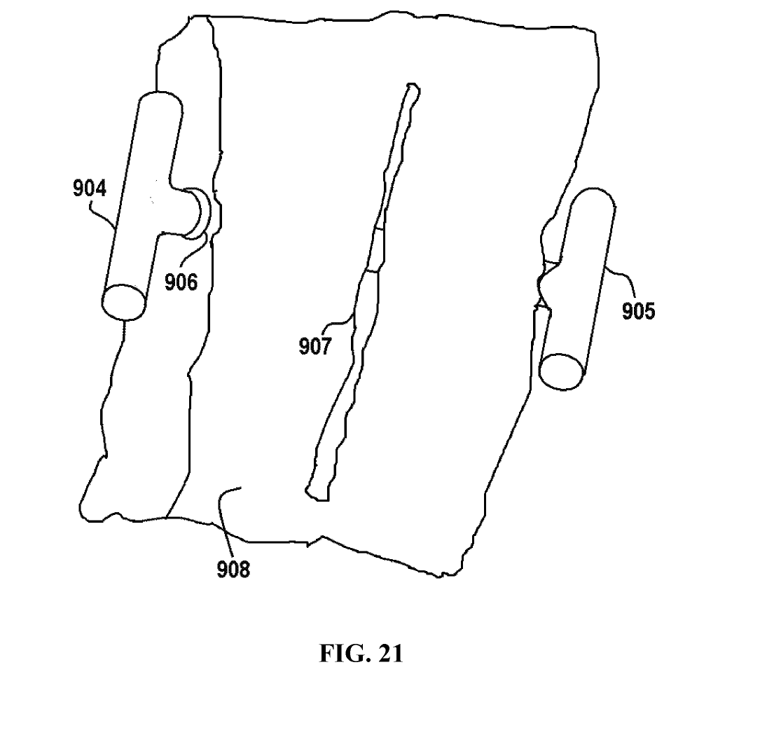

In the embodiment shown in FIG. 20B, fastening member 905 is located at an angle “A” that is less than approximately 45 degrees (or less) to body 901. In certain embodiments, fastening member 905 may be moved so that it is approximately parallel to body 901. Referring now to FIG. 21, during use fastening member 905 can be moved so that the angle between fastening member 905 and body 901 is minimized. Fastening member 905 can then be inserted through an incision 906 on one side of an opening 907 (e.g., a cut, tear, hole, etc.) in tissue 908 and pushed through to the other side of opening 907. Fastening member 905 can then be passed through an incision (not visible) on the other side of opening 907. As shown in the exemplary embodiment of FIG. 21, with fastening members 904, 905 anchored on opposite sides of opening 907, tissue 908 is held together to assist in closing opening 907 and expediting healing of tissue 908.

Referring now to FIG. 22, the illustrated fastening device 920 is similar to the previously-described fastening device 900. In this embodiment, however, the body is a two-piece design that can be separated for installation. As shown in the exemplary embodiment, fastening device 920 comprises a first elongate body 921 with a first end 922 and a second end 924. A first fastening member 928 is coupled to first elongate body 921 proximal to first end 922 and a penetrating member 925 is coupled proximal to second end 924. Fastening device 920 also comprises a second elongate body 923 having a first end 926 and a second end 928. A second fastening member 929 is coupled to second elongate body 923 proximal to first end 926 and a receiving member 927 is coupled proximal to second end 924.

During use, penetrating member 925 can be extended through the tissue, the two components of first and second elongate bodies 921, 923 can be re-coupled so that they appear similar to the configuration shown in FIG. 21. Fastening members 928 and 929 can be disposed on opposite sides of a tissue opening and can assist in closing a tissue opening and expediting healing of the tissue. First and second elongate bodies 921, 923 may be coupled in any suitable manner. In certain embodiments, for example, first and second elongate bodies 921, 923 may be press fit together via an interference fit between penetrating member 925 and receiving member 927.

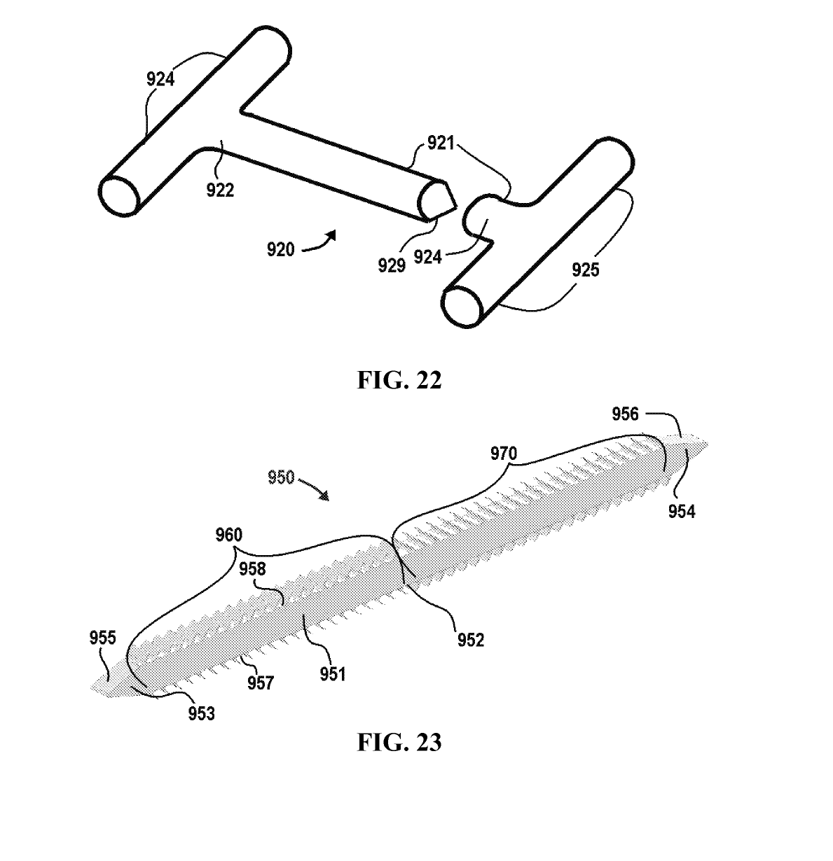

Referring now to FIG. 23, an exemplary embodiment of a fastening device 950 comprises an elongate body 951 comprising a central portion 952, a first end 953, and a second end 954. Fastening device 950 also comprises a first penetrating member 955 proximal to the first end 953 and a second penetrating member 956 proximal to the second end 954. Fastening device further comprises a first plurality of engagement members 960 coupled to elongate body 951 between central portion 952 and first end 953. Fastening device also comprises a second plurality of engagement members 970 coupled to elongate body 951 between central portion 952 and second end 954.

In the embodiment shown in FIG. 23, first plurality of engagement members 960 are angled away from first end 953 (e.g., as an engagement member 960 extends away from body 951, engagement member 960 angles closer to central portion 952 and away from first end 953). Similarly, second plurality of engagement members 970 are angled away from second end 954 (e.g., as an engagement member 970 extends away from body 951, engagement member 970 angles closer to central portion 952 and away from second end 954). In the embodiment shown, body 951 comprises a square (or rectangular) cross-section with a first side 957 opposing a second side 958. Engagement members 960 and 970 can be disposed on both first side 957 and second side 958.

During use, first penetrating member 955 may pierce one side of an opening in tissue and be inserted into the tissue so that engagement members 960 engage the tissue on one side of the opening. In addition, second penetrating member 956 may pierce another side of the tissue opening and be inserted into the tissue so that engagement members 970 engage the tissue on that side of the opening. The tissue on each side of the opening can then be drawn towards center portion 952 to bring the tissue closer together. Engagement members 960 and 970 are configured to hold the tissue in place to assist in closing the tissue opening and expediting healing of the tissue. The angled configuration of engagement members 960 and 970 restricts movement of tissue away from center portion 952 to assist in the healing process.

### Equivalents and Scope

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that embodiments of the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, embodiments of the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

1. A fastening device for fastening tissue, the fastening device comprising:
a body that forms a partial ring shape, wherein the body comprises a first end and a second end;
a first fastening member proximal to the first end of the body, wherein the first fastening member comprises an aperture; and
a second fastening member proximal to the second end of the body, wherein the second fastening member comprises a plurality of engagement members configured to be inserted through the aperture.

2. The fastening device of claim 1 wherein the plurality of engagement members are spaced along the body.

3. The fastening device of claim 1 wherein the plurality of engagement members are configured to restrict the second end from being withdrawn from the aperture when an engagement member is inserted through the aperture.

4. The fastening device of claim 1 wherein the body forms a ring shape when an engagement member is inserted through the aperture.

5. The fastening device of claim 4 wherein the plurality of engagement members are configured to allow the ring shape to be contracted but not expanded when an engagement member is inserted through the aperture.

6. The fastening device of claim 1 wherein the engagement members and the aperture form a ratcheting mechanism.

7. The fastening device of claim 1 wherein the body comprises a non-circular cross-section.

8. A method of fastening tissue, the method comprising:
providing a fastening device according to claim 1;
approximating a first portion of tissue to a second portion of tissue;
piercing the first portion of tissue and the second portion of tissue with the second fastening member; and
inserting an engagement member through the aperture of the first fastening member.

9. A fastening device for fastening tissue, the fastening device comprising:
a first suture comprising a first fastening member;
a second suture comprising a second fastening member; and
a coupling member coupling the first suture to the second suture, wherein the coupling member is configured to slide relative to the first and second suture.

10. The fastening device of claim 9 wherein the coupling member comprises a first aperture and a second aperture, and wherein the first suture extends through the first aperture and the second suture extends through the second aperture.

11. The fastening device of claim 9 wherein the first fastening member is coupled to a first end of the first suture and wherein the second fastening member is coupled to a first end of the second suture.

12. The fastening device of claim 9 wherein the first fastening member and the second fastening member each comprise a pointed end configured to penetrate tissue.

13. A method of fastening tissue, the method comprising:
providing a fastening device according to claim 9;
approximating a first portion of tissue to a second portion of tissue;
piercing the first portion of tissue with the first fastening member;
piercing the second portion of tissue with the second fastening member; and
moving the coupling member toward the first and second fastening members.

14. The method of claim 13 further comprising:
tying a first knot in the first suture so that the coupling member is positioned between the first knot and the first fastening member; and
tying a second knot in the second suture so that the coupling member is positioned between the second knot and the second fastening member.

15. The method of claim 13 further comprising tying the first suture and the second suture in a knot so that the coupling member is positioned between the knot and the first and second fastening members.

16. A fastening device for fastening tissue, the fastening device comprising:
an elongate body with a first end and a second end;
a first fastening member proximal to the first end of the elongate body; and
a second fastening member proximal to the second end, wherein:
the second fastening member is configured to move from a first position where the second fastening member is located at a first angle to the elongate body to a second position wherein the second fastening member is located at a second angle to the elongate body, and wherein the first angle is greater than the second angle.

17. The fastening device of claim 16, wherein the first angle is approximately 90 degrees.

18. The fastening device of claim 16, wherein the second angle is less than 45 degrees.

19. A method of fastening tissue, the method comprising:
providing a fastening device according to claim 16;
approximating a first portion of tissue to a second portion of tissue;
placing the second fastening member in the second position of claim 16;
inserting the second fastening member through an incision in the first portion of tissue and the second portion of tissue; and
placing the second fastening member in the first position of claim 16.

20. A fastening device for fastening tissue, the fastening device comprising:
a first elongate body with a first end and a second end;
a first fastening member proximal to the first end of the first elongate body and a penetrating member proximal to the second end of the first body;
a second elongate body with a first end and a second end;
a second fastening member proximal to the first end of the second elongate body and a receiving member proximal to the second end of the second elongate body.

21. The fastening device of claim 20 wherein the penetrating member and receiving member are configured for an interference fit.

22. A method of fastening tissue, the method comprising:
providing a fastening device according to claim 20;
approximating a first portion of tissue to a second portion of tissue;
inserting the penetrating member through the first and second portions of tissue; and
coupling the first elongate body and the second elongate body by inserting the penetrating member into the receiving member.

23. A fastening device for fastening tissue, the fastening device comprising:
an elongate body comprising a central portion, a first end and a second end;
a first penetrating member proximal to the first end;
a second penetrating member proximal to the second end;
a first plurality of engagement members coupled to the elongate body between the central portion and the first end, wherein the first plurality of engagement members are angled away from the first end; and
a second plurality of engagement members coupled to the elongate body between the central portion and the second end, wherein the second plurality of engagement members are angled away from the second end.

24. The fastening device of claim 23 wherein the elongate body comprises a first side and a second side, and wherein the first plurality of engagement members are disposed on the first side and the second side of the elongate body.

25. The fastening device of claim 23 wherein the second side is opposing the first side.

26. A method of fastening tissue, the method comprising:
providing a fastening device according to claim 23;
approximating a first portion of tissue to a second portion of tissue;
inserting the first penetrating member into the first portion of tissue; and
inserting the second penetrating member into the second portion of tissue.

27. A fastening device according to claim 1, 9, 16, 20, or 23, wherein the fastening device comprises an elastomeric material.

28. The fastening device of claim 27 wherein the elastomeric material is a polyhydroxyalkanoate material.

29. The fastening device of claim 27 wherein the elastomeric material is combined with a crystalline or amorphous polymer.

30. The fastening device according to claim 27 wherein the fastening device has an elasticity equivalent to that of mucosal tissue.

31. A fastening device according to claim 1, 9, 16, 20, or 23, wherein the fastening device comprises a shape memory material.
